(19) United States
(12) Patent Application Publication
(10) Pub. No.: US 2011/0021414 A1
(43) Pub. Date: Jan. 27, 2011

(54) Chimeric Phage Tail Proteins and Uses Thereof

(75) Inventors: Ryland F. Young, College Station, TX (US); Douglas K. Struck, College Station, TX (US)

Correspondence Address: WINSTEAD PC, P.O. BOX 50784, DALLAS, TX, 75201, US

(73) Assignee: Texas A & M University System, College Station, TX (US)

(21) Appl. No.: 12/739,915

(22) PCT Filed: Oct. 24, 2008
(86) PCT No.: PCT/US08/81151

§ 371 Date: Oct. 1, 2010

Related U.S. Application Data
(60) Provisional application No. 60/982,371, filed on Oct. 24, 2007.

(51) Int. Cl. A01N 63/02; C12N 7/00; C07K 14/005; C12N 15/63; C12N 1/21; A01P 1/00

(52) U.S. Cl. 514/2.4; 435/235.1; 530/350; 435/320.1; 435/252.3

(57) ABSTRACT

A multi-protein pyocin-like structure derived from a bacteriophage or a bacteriocin that includes a chimeric tail fiber having a protein receptor binding domain capable of recognizing Lipid A. A multiprotein pyocin-like structure, derived from a bacteriophage or a bacteriocin that includes a chimeric tail fiber capable of recognizing and binding MurNac-L-Ala-D-Glu. A phage including a chimeric tail fiber that binds Lipid A. A plasmid encoding a chimeric ail fiber that binds MurNac-L-Ala-D-Glu. A plasmid encoding a chimeric tail fiber that binds Lipid A. A chimeric bacteriocin or bacteriophage derived tail fiber that includes the amino terminal of the human bactericidal/permeability-increasing protein (BPINTD)—A chimeric bacteriophage or bacteriocin derived tail fiber also includes a binding domain encoding the mammalian Nod2cτD—An antibacterial agent includes a bacteriocidal/permeability-increasing protein receptor domain that binds to Lipid A. An antibacterial agent also includes Nod2 carboxyl-terminus receptor domain that binds MurNac-L-Ala-D-Glu.

### RELATED APPLICATION

The present application claims the benefit of co-pending U.S. Provisional Patent Application No. 60/982,371, filed Oct. 24, 2007, which is incorporated herein by reference in its entirety.

### BACKGROUND OF THE INVENTION

Disease-causing microbes that have become resistant to drug therapy are an increasing public health problem. Such antimicrobial resistance or drug resistance, is due largely to the increasing use of antibiotics. Additionally, new antibotics are having trouble keeping up with the problem, even though new approaches may be generated based on the flow of structural information for bacterial proteins or from the ability to use robotic screens to sift through enormous chemical compound libraries for effective bactericidals.

The increased prevalence of antibiotic resistance is an outcome of evolution. All organisms, bacteria included, naturally include variants. Upon undergoing a selective pressure, a resistant population emerges. This renegade population then multiplies leading to the variant becoming the predominant organism. Though bacterial antibiotic resistance is a natural phenomenon, societal factors also contribute to the problem. Several studies have demonstrated that resistant organisms are a result of patterns of antibiotic usage. Other factors contributing towards resistance include incorrect diagnosis, unnecessary prescriptions, and improper use of antibiotics by patients. Additionally, antibiotic drugs given to food-producing animals for important therapeutic, preventative, or production reasons cause microbes to become resistant to drugs used to treat human illnesses, thereby making human illnesses harder to treat.

In light of the foregoing, there is a continuing need to develop new strategies for combating resistant bacteria and different strategies are needed to ameliorate the problem of rapid developing bacterial resistance.

### SUMMARY OF THE INVENTION

Provided herein is a nucleic acid molecule encoding a multi-protein structure comprising a phage with a chimeric tail fiber protein engineered to replace at least a portion of the endogenous receptor binding domain with a protein domain capable of binding an invariant receptor present on the surface of a bacterial cell envelope. Alternatively, provided is a nucleic acid molecule encoding a multi-protein structure comprising a headless lysis-defective phage with a chimeric tail fiber protein engineered to replace at least a portion of the endogenous receptor binding domain with a protein domain capable of binding an invariant receptor present on the surface of a bacterial cell envelope wall.

Also, provided is a nucleic acid molecule encoding a chimeric phage tail fiber protein, where the tail fiber protein is engineered to replace at least a portion of the endogenous receptor binding domain with a protein domain capable of binding an invariant receptor present on the surface of a bacterial cell envelope.

Additionally, provided is a vector capable of expressing the nucleic acids described above, where the vector comprises the nucleic acid and regulatory elements necessary for expression of the nucleic acid. An isolated bacterial cell infected with the vector described above is also disclosed herein. Additionally, the bacterial cell infected with the vector described above may harbor a prophage or a pyocin, where the tail fiber genes of the prophage or pyocin are inactivated.

Also, disclosed is a method of producing an anti-microbial agent, the method comprising culturing the bacterial cell described above under conditions favoring the growth of the cell.

Furthermore, provided is an anti-microbial agent comprising a multiprotein structure encoded by the nucleic acids described above.

Additionally, disclosed is an antimicrobial agent comprising a phage tail fiber protein, where at least a part of the receptor binding domain of the phage tail fiber protein is the amino-terminal of the bacteriocidal/permeability-increasing protein.

An antimicrobial agent comprising a phage tail fiber protein where at least a part of the receptor binding domain of the phage tail fiber protein is the carboxyl terminal of the Nod2 receptor protein is disclosed herein.

Provided also is a phage tail fiber protein, where at least a part of the receptor binding domain is the amino terminus of the bacteriocidal/permeability-increasing protein. Additionally, provided is a phage tail fiber, where at least a part of said receptor binding domain is the carboxyl terminus of the Nod2 protein.

A method of reducing a bacterial population disposed on a surface comprising exposing the surface to the anti-microbial agent described above is also provided.

Provided also is a method of reducing a bacterial population in an aqueous environment comprising adding the anti-microbial agent of claim 16 to the aqueous environment.

A novel antibacterial agent is provided herein comprising a chimeric phage tail protein where at least a portion of the endogenous receptor binding domain is replaced with the bacteriocidal/permeability-increasing protein receptor domain that binds to bacteriocidal/permeability-increasing protein (Lipid A). Alternatively, the chimeric phage tail protein disclosed herein is such that at least a portion of the endogenous receptor binding domain is replaced with the Nod2_{CTD} receptor domain that binds MurNac-L-Ala-D-Glu. Likewise, a phage that includes a chimeric tail fiber that binds MurNac-L-Ala-D-Glu or Lipid A is also provided as a novel anti-bacterial agent.

The foregoing has outlined the features of various embodiments in order that the detailed description that follows may be better understood. Additional features and advantages of various embodiments will be described hereinafter which form the subject of the claims of the invention.

### BRIEF DESCRIPTION OF THE DRAWINGS

The foregoing summary as well as the following detailed description of the preferred embodiment of the invention will be better understood when read in conjunction with the appended drawings. It should be understood, however, that the invention is not limited to the precise arrangements and instrumentalities shown herein.

For a more complete understanding of the present invention, and the advantages thereof, reference is now made to the following descriptions taken in conjunction with the accompanying drawings, in which:

FIGS. 1A-1B show Bacteriocin(A) and Pyocin-like multi-protein structure (B) particles from B. cenocepacia.

FIG. 2 depicts Gram-negative envelope. The outer leaf of the outer membrane is LPS, composed of the variable polysaccharide O-antigen, variable, species specific polysaccharide core and the conserved lipid A. MDO=membrane-derived oligosacchrides, kdo 3-deoxy-d-manno-oct-2-ulosonic acid; PPEtn=phosphoethanolamine.

FIG. 3 shows chemical structure of Lipid A, a beta-(1-6)-linked glucosamine disaccharide with ester and amide-linked fatty acids.

FIG. 4 shows bacteriocin structure adsorbed on the surface of a bacterial cell wall.

FIG. 5 shows Myophage and Siphophage morphology.

FIGS. 6A-6B show crystal structure of the human BPI (FIG. 6A) and secondary structure of 23 kDa amino-terminal Lipid A binding domain of BPI (FIG. 6B).

FIG. 7 shows Gram-positive cell wall structure.

FIG. 8 shows peptidoglycan subunit of the gram-positive cell wall with the Nod2 recognition moiety.

FIG. 9 shows crystal structure of carboxyl-terminal domain of the human PGRP-1a, a close homolog of Nod2 bound to a muramyl peptide.

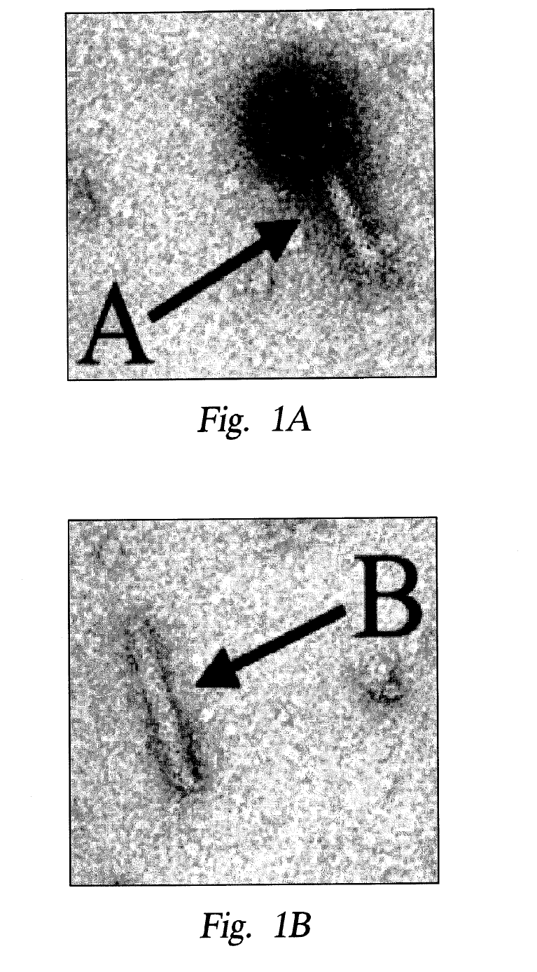

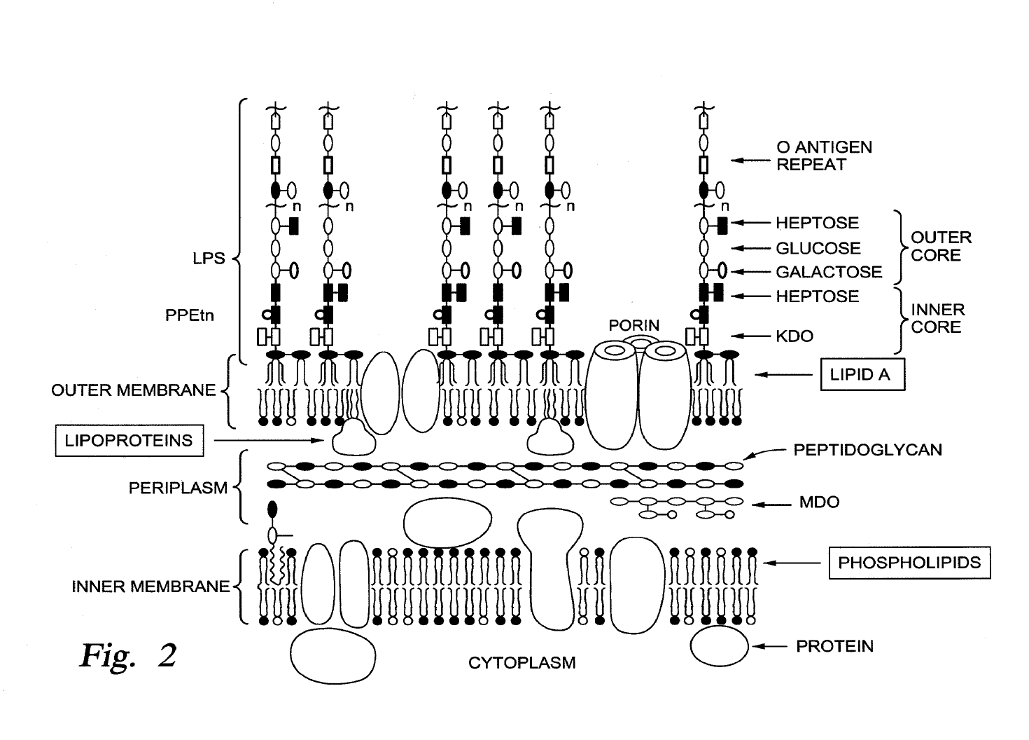

### DETAILED DESCRIPTION OF THE INVENTION

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition.

As used herein, bacteriocin includes an R-type pyocin, tail-like bacteriocin, R-type bacteriocin, F-type and R-type pyocins, monocins, meningocins, or other bacteriocins. A bacteriocin includes modified versions of R-type and F-type pyocins, enterocoliticins, monocins, and meningocins (see Kingsbury “Bacteriocin production by strains of Neisseria meningitidis.” J Bacteriol. 91(5):1696-9, 1966). A modified or engineered bacteriocin may be a modified R-type pyocin selected from the R1, R2, R3, R4, or R5 pyocin of P. aeruginosa. An additional property common to bacteriocins and engineered bacteriocins disclosed herein is that they do not contain nucleic acid and thus are replication deficient such that they cannot reproduce themselves after or during the killing of a target bacterium as can many bacteriophages.

Bacteriocins disclosed herein, are complex molecules comprising multi-protein structures, or polypeptide, subunits that resemble the tail structures of bacteriophages of the myoviridae family or are pyocin-like structures. In a naturally occurring bacteriocin (pyocins), the subunit structures are encoded by the bacterial genome, such as that of Pseudomonas aeruginosa, and form pyocins to serve as natural defenses against other bacteria (Kageyama, 1975). A sensitive, target bacterium can be killed by a single pyocin molecule (Kageyama, 1964; Shinomiya & Shiga, 1979; Morse et al., 1980; Strauch et al., 2001).

A “target bacterium” or “target bacteria” refer to a bacterium or bacteria that are bound by an engineered hmw bacteriocin of the disclosure and/or whose growth, survival, or replication is inhibited thereby. The term “growth inhibition” or variations thereof refersto the slowing or stopping of the rate of a bacteria cell's division or cessation of bacterial cell division, or to death of the bacteria.

The terms “polypeptide”, “peptide”, and “protein” are typically used interchangeably herein to refer to a polymer of amino acid residues. Amino acids may be referred to herein by either their commonly known three letter symbols or by the one-letter symbols recommended by the IUPAC-IUB Biochemical Nomenclature Commission.

As used herein, a “nucleic acid” typically refers to deoxyribonucleotide or ribonucleotides polymers (pure or mixed) in single- or double-stranded form. The term may encompass nucleic acids containing nucleotide analogs or modified backbone residues or linkages, which are synthetic, naturally occurring, and non-naturally occurring, which have similar binding, structural, or functional properties as the reference nucleic acid, and which are metabolized in a manner similar to the reference nucleotides. Non-limiting examples of such analogs include, without limitation, phosphorothioates, phosphoramidates, methyl phosphonates, chiral-methyl phosphonates, 2-0-methyl ribonucleotides, and peptide-nucleic acids (PNAs). The term nucleic acid may, in some contexts, be used interchangeably with gene, cDNA, mRNA, oligonucleotide, and polynucleotide.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention.

In an embodiment, the present disclosure provides an antibacterial agent or peptide that is a multi-protein structure derived from a phage or a bacteriocin. The multi-protein structure includes a polypeptide encoded by a recombinant bacteriophage or a bacteriocin gene with a chimeric tail fiber, where the tail fiber is engineered to replace the receptor binding domain with a protein domain able to recognize and bind an essential, universal, conserved, and immutable non-protein/motif exclusive to the bacterial cell wall. Some examples of bacteriocins include but are not limited to colicins, monocins, meningocins and pyocins. The multi-protein structure disclosed herein includes, but is not limited to lysis-defective phage with a contractile tail and a recombinant lysogeny-defective temperate phage with contractile tail or engineered to express contractile tail. Examples of temperate phages include but are not limited to myophages, P2 and Mu. Non-proteins/motifs exclusive to bacterial cell wall include but are not limited to those exclusively present in the gram-negative bacterial cell wall and in the gram-positive bacterial cell wall. The protein domains include but are not limited to those recognizing and binding Lipid A and monosaccharide dipeptide L-alanyl-D-glutamyl-N-acetylmuramic acid (MurNac-L-Ala-D-Glu).

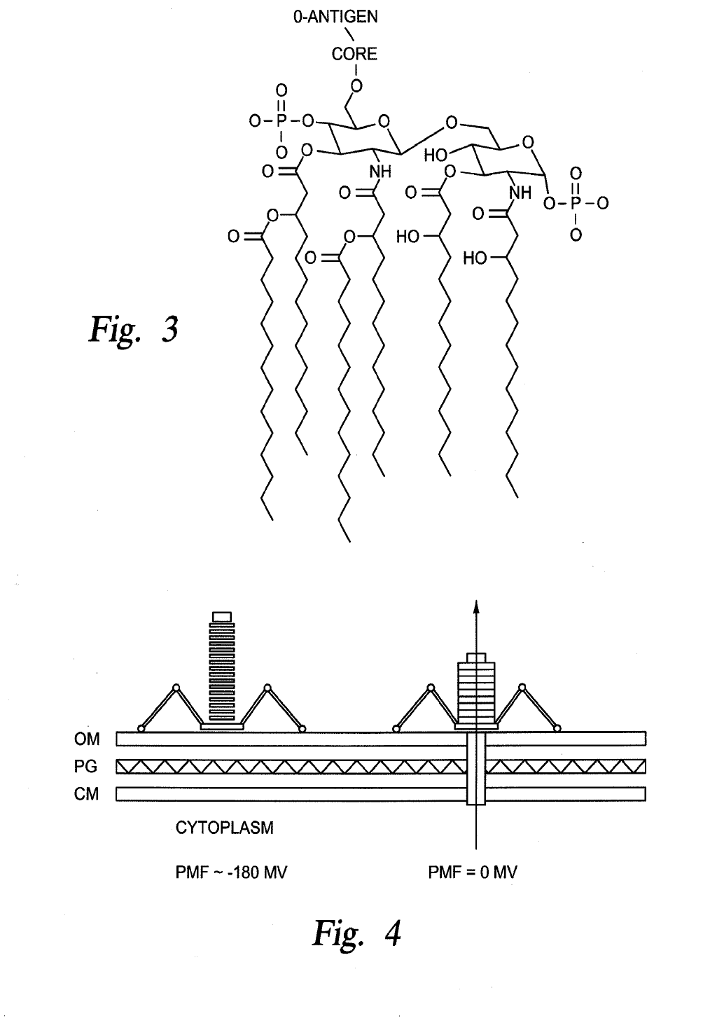

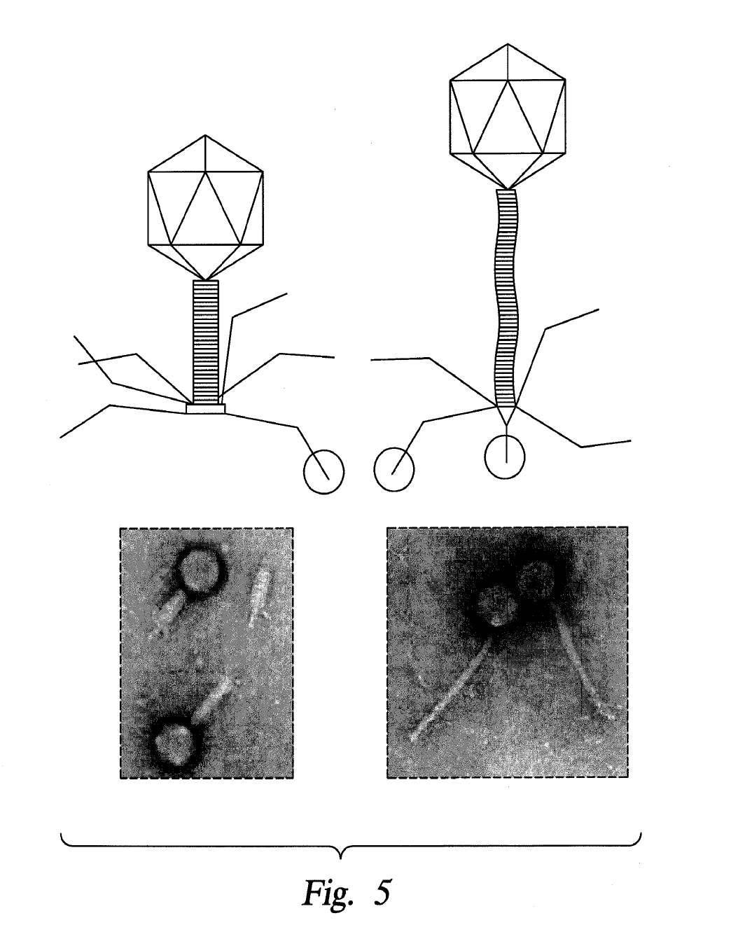

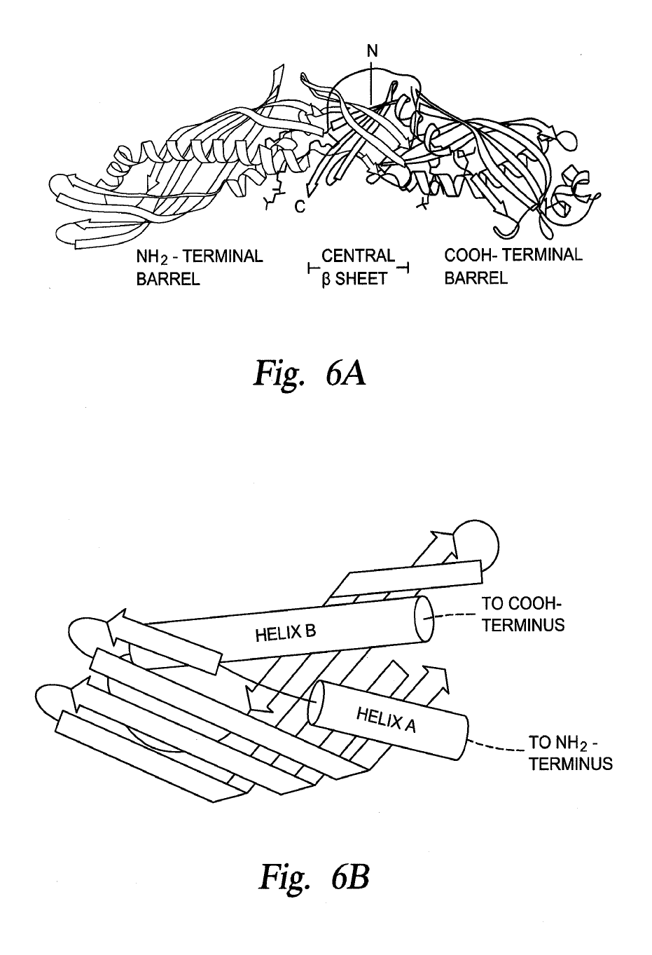

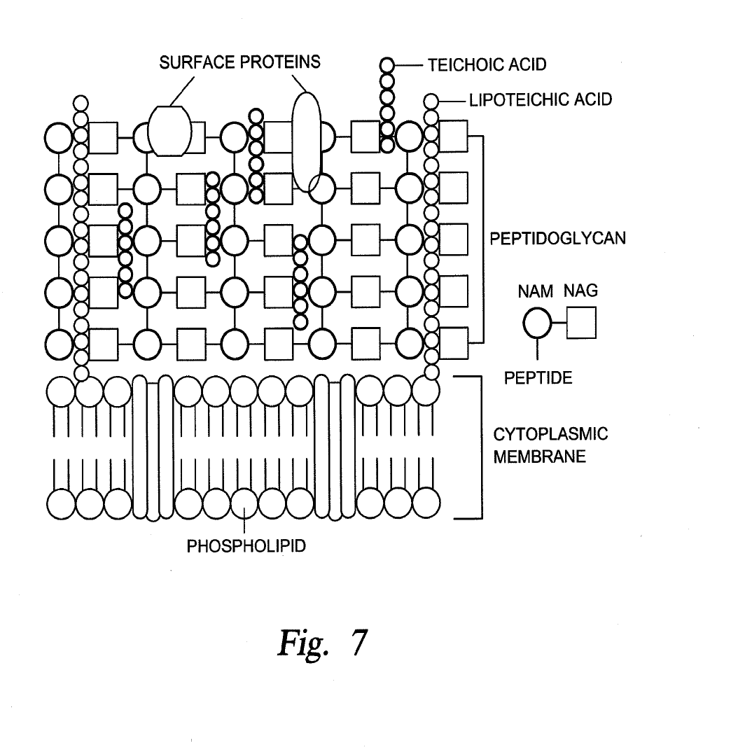

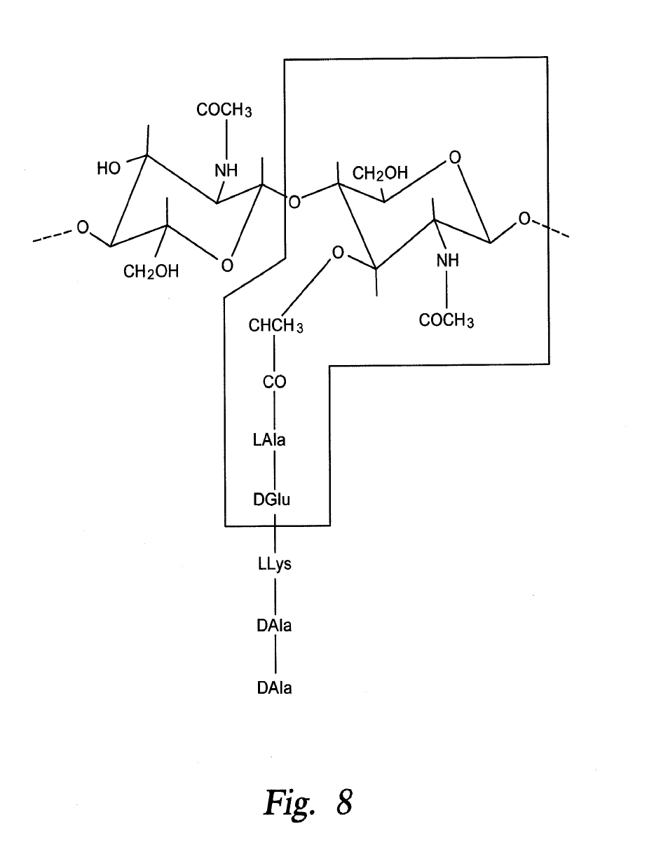

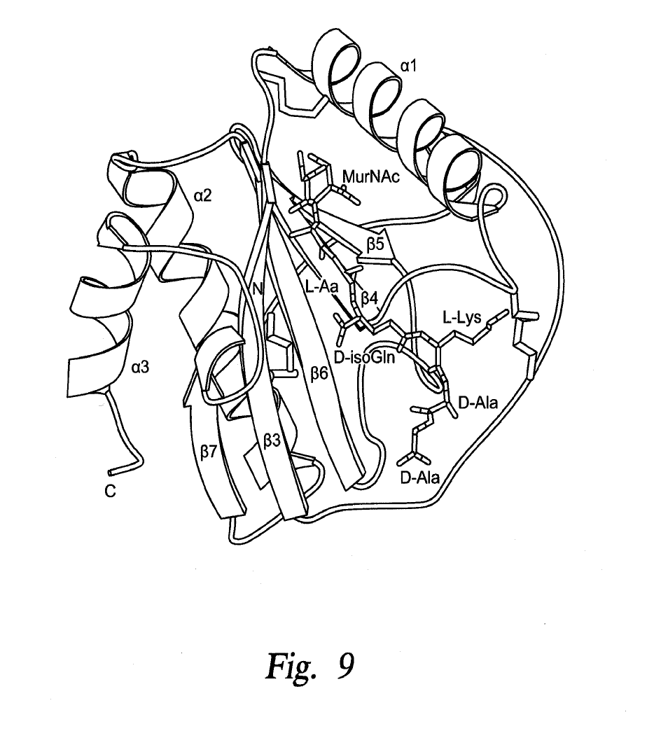

In a related embodiment, the multi-protein structures resemble a bacteriophage (or phage) in morphology, except that they lack the head group containing the genetic information used in phage replication (cf. FIG. 3 and FIG. 4, Appendix A). The structures described herein are essentially “headless” bacteriophage particles. These antimicrobial peptides represent an example of a broader class of proteins produced by many bacteria, called bacteriocins. Bacteriocins refer to any protein structure or peptide obtained from a bacteria that is capable of killing other bacteria. Bacteriocins can be broad or narrow spectrum and are not lethal to the cells which produce them. Bacteria protect themselves from the lethal effects of their own bacteriocins by such mechanisms as post-translational modification or the production of an immunity protein(s). In any case, bacteriocins are potent antibacterial substances produced by a large and diverse assortment of species. The first type of such multi-protein structures occurring in nature were found in Pseudomonas strains, called pyocins, and were among several types of bacteriocins that were produced by these bacteria (Michel-Briand, Y. & Baysse, C. (2002) The pyocins of Pseudomonas aeruginosa, Biochimie 84, 499-510). Similarly, E. coli produces analogous multi-protein structures called colicins.

Like bacteriophages (phages), the bacteriocins adsorb to specific features on the surface of the target cell via a receptor binding domain (RBD), present in the tail fiber, which binds to, or interacts with, a receptor to form a binding pair. After irreversible adsorption, the first step of the infection process involves forming a hole in the cytoplasmic membrane. For phages, the DNA is introduced through the hole, which is then sealed to allow for the infection cycle to begin. Unlike phages, however, bacteriocins perform only the hole-forming step. The resulting hole is permanent and kills the bacteria as a result of leakage of vital bacterial molecules like potassium, amino acids, and other low-molecular-weight molecules from sensitive cells, via the open core.

In another embodiment, the multi-protein structure equipped with the recombinant tail fibers having a receptor binding domain for Lipid A is considered a universal anti-bacterial agent that can kill a very broad range of Gram-negative bacteria. Lipid A is the base structure of lipopolysachamide (LPS), and occurs in the cell wall of all Gram-negative bacteria. Lipid A is the perfect receptor for phages and bacteriocins to target, because it is universal and immutable. Mutations that block lipid A synthesis would be lethal to the bacterial cell.

The multi-protein structure possessing the recombinant tail fiber may be derived from pyocin or colicin structures, for example. Any naturally occurring multi-protein structure possessing the phage-like morphology described above may be engineered to incorporate a receptor binding domain for Lipid A. There are two main types of pyocins that are morphologically indistinguishable from the tail structures of bacteriophages: R-type multi-protein structures and F-type multi-protein structures. R-type multi-protein structures have contractile tails and constitute the base structures for development of a universal multi-protein structure contemplated herein. A universal multi-protein structure has the ability to bind to and kill any Gram-negative bacterium with unit efficiency. (i.e., one multi-protein structure will kill one cell) based on the R-type pyocin. Finally there is a third type of pyocins described as a soluble pyocins or S-type pyocin that may be similarly genetically altered with the recombinant tail fiber.

The multi-protein structures kill by adsorbing to the surface of a bacterial cell and initiating an injection process. Because the multi-protein structures are incomplete bacteriophage particles, the injection process does not go beyond the formation of a lethal hole in the cytoplasmic membrane of the host cell. Multi-protein structures in nature have host ranges that vary from extremely narrow (i.e., only specific other strains in the same species) to broad within a bacterial species and occasionally to other species, but no multi-protein structure has been found that is lethal to many different genera of bacteria.

The protein binding domain to be incorporated into the receptor binding domain of the tail fiber for a universal multi-protein structure may be the N-terminal domain of the bactericidal/permeability-increasing protein (BPI). BPI is a protein found in human granulocytes that binds Lipid A. This sequence may be used as a supplement to the naturally occurring protein binding domain or as a replacement. A 23 kDa N-terminal domain (“BPI_{NTD}”) fragment consisting of approximately the first 200 amino acids is known to have the Lipid A binding activity [Appelmelk, B. J. et al. (1994) Recombinant human bactericidal/permeability-increasing protein (rBPI23) is a universal lipopolysaccharide-binding ligand, Infect. Immun. 62, 3564-3567]. In the concept of the universal multi-protein structure, the DNA sequence encoding BPI_{NTD} may be used to replace all or part of the DNA encoding the receptor-binding domain in the tail fiber gene of a multi-protein structure. Additionally, other BPI from other mammalian systems may be used in the recombinant tail fiber. In another embodiment, the BPI region may also be genetically modified as well. In yet another embodiment other protein binding domains capable of recognizing Lipid A may be used.

As stated previously, the universal multi-protein structure may be based on the R-type pyocins; i.e., a multi-protein structure with a contractile tail. These are morphologically indistinguishable from the contractile tails of the subgroup of the Caudovirales (tailed bacteriophages) called Myoviridae; for the purposes of this invention, phages with contractile tails will be called myophages. In R-type pyocins, and in myophages, bacteriophages that have the morphology of Myoviridae, the specificity determinants are found in the six long tail fibers that are attached to the base-plate of the tail. A single tail fiber gene encodes the specificity determinant, the location of which has been mapped in a few Myoviridae. The precise location of the specificity determinants within the tail fiber gene can be mapped by standard genetic and molecular techniques, and by bioinformatic comparisons of tail fiber genes from different Myoviridae and R-type multi-protein structures. In principle any multi-protein structure with a single gene specifying a long tail fiber that has the host specificity determinant could be used. Another version would begin with an intact myophage prophage; e.g., the coliphage P2 or the coliphage Mu. In this form of the universal multi-protein structure, the engineering of the tail fiber gene will be done in the context of the prophage, which will allow for convenient genetic manipulation, after which the head genes will be deleted or inactivated to generate a multi-protein structure gene.

Another aspect of the multi-protein structure concept is that the lysis system of the multi-protein structure may be inactivated. This may be beneficial because multi-protein structures are produced by spontaneous induction of a sub-population of the bacteria carrying the multi-protein structure genes. The induction process results in expression of the multi-protein structure genes, formation of multi-protein structure particles, and lysis of the induced cell, releasing the multi-protein structures to the medium. In the universal multi-protein structure, there is a benefit from deleting the lysis genes because the free multi-protein structures would otherwise bind to and kill the cells carrying the multi-protein structure determinant. In the universal multi-protein structure concept, the lysis genes that may be deleted or inactivated are, at minimum, the holin gene, and also the endolysin gene if the latter encodes a secretory endolysin. Inactivation of the holin gene has the additional advantage of greatly increasing the production of multi-protein structures in a cell, compared to the parental multi-protein structure, because the holin function is to terminate the induction at a programmed time, and cause lysis by permeabilizing the host membrane.

The present disclosure also provides, in a related embodiment, a phage that includes a recombinant tail fiber that binds Lipid A. Complete myophage bacteriophages may be generated by replacing or supplementing the determinants in their tail fibers with BPI_{NTD}. These modified myophages will have the same ability to bind to all Gram-negative cells. These modified myophages will thus have broad host ranges, although not as broad as the universal multi-protein structure. The reason for this is that in the process of infection, bacteriophages inject their DNA into the cells, and then effect closure of the membrane hole that is used to transfer the DNA. In cells that are significantly diverged from the original host of the bacteriophage, there may be many incompatibilities between the genes and proteins of the bacteriophage and the host cell cytoplasm, so in these cases infection cycles may not be successful. Nevertheless, a myophage with the engineered specificity determinants carrying the BPI_{NTD} domain will have much broader specificity than the original myophage. This engineering could be applied to myophages of many different Gram-negative bacteria.

In yet, another embodiment the present disclosure provides a phage that includes a recombinant tail fiber that binds peptidoglycan motif MurNac-L-Ala-D-Glu. This represents a potentially universal phage against Gram-positive bacteria. In such a construct, the recombinant tail fiber may include a binding domain of human protein Nod2_{CTD}, for example. The recombinant tail fiber may also include a binding domain of a member of the Pattern Recognition Receptors (PRR) superfamily.

It is known that for the Myoviridae, phages with contractile tails, the specificity determinant resides in the carboxy-terminal end of the long tail fiber proteins. These protein domains recognize and bind to the phage receptors, which are specific elements on the surface of bacteria. For Gram-positive bacteria, phage-receptors are either elements of the peptidoglycan or surface proteins. Different phages use different receptors, and most receptors are non-essential surface features that can be changed by mutations in the bacterium. These considerations severely limit the feasability of using phages for therapeutic, prophylactic, and diagnostic purposes, because for any given target species of bacterial pathogen, it will take multiple phages to recognize all the potential clinical isolates. The invention would equip phages with the ability to recognize an essential, non-proteinaceous component of the Gram-positive cell wall and thus eliminate or greatly reduce the need for more than one phage per pathogenic species.

In still yet another embodiment the present disclosure provides a method to construct recombinant tail fibers in which the less than 250 amino acids C-terminal domain of the human protein Nod2 (Nod2_{CTD}), or one of its homologs in the Pattern Recognition Receptors (PRR) family, is inserted into the C-terminal half of the long tail fiber of the phage. Nod2 is a protein of the innate immunity system which recognizes the only invariant moeity in Gram-positive murein: the sugar-peptide MurNac-L-Ala-D-Glu (see Girardin et al., 2003, Appendix A). The normal receptor-binding domain may or may not be deleted in different versions of the invention. The result is that a phage with such recombinant tail fibers will be able to bind to the surface of any Gram-positive cell in which the murein is solvent-exposed.

Nucleic acids, vectors, and bacterial cells may be used in a method of producing a modified or engineered phage or bactericin as disclosed herein. Such a method may comprise culturing bacterial cells containing nucleic acid molecules under conditions resulting in the expression and production of the tail fiber and bacteriocin.

The insertion may be constructed by recombinant DNA technology in a plasmid clone of the tail fiber gene of each particular phage, with the requirement that the phage be a contractile-tail phage (Myovirus). The recombinant phage tail fiber gene may be expressed constitutively from the plasmid carried in a cell that is susceptible to the phage. Growth of the phage in a culture of the plasmid-containing cell will result in phage particles that have some or all of its tail fibers replaced by the recombinant tail fibers containing the inserted Nod2_{CTD}. Alternatively, the plasmid carrying the recombinant tail fiber gene may be carried in a cell containing a prophage. Induction of the prophage will result in the production of virions that have some or all of their tail fibers replaced by the recombinant tail fibers containing the inserted Nod2_{CTD}.

Any of the constructs for modifying tail fibers of any multi-protein structures or phages may be expressed in a plasmid or other appropriate vector. In particular, a plasmid may have a recombinant tail fiber gene that encodes a tail fiber that binds MurNac-L-Ala-D-Glu or a tail fiber gene that encodes a tail fiber that binds Lipid A. The term “tail fiber” may refer to either the oligonucleotide sequence that encodes the protein or the protein itself. Thus, the present disclosure also provides tail fibers that include a binding domain of a bacteriocidal/permeability-increasing protein as well as tail fibers that include a binding domain of Nod2_{CTD}.

Finally, the present disclosure provides antibacterial agents constructed by including a bacteriocidal/permeability-increasing protein receptor domain that binds to Lipid A or Nod2_{CTD} receptor domain that binds MurNac-L-Ala-D-Glu within its structure.

### PROPHETIC EXAMPLES

The following prophetic examples are included to demonstrate particular embodiments. It should be appreciated by those of skill in the art that the methods disclosed in the examples that follows merely represent exemplary embodiments. However, those of skill in the art should, in light of the present disclosure, appreciate that many changes can be made in the specific embodiments described and still obtain a like or similar results.

### Example 1

### Redirecting the Pseudomonas R2 Pyocin

One approach is to re-engineer an existing pyocin-like multi-protein structure to recognize Lipid A as a receptor. The best candidates are the original multi-protein structures, the pyocins of Pseudomonas. There are two types of phage-tail-like pyocins, the R pyocins and F pyocins. R pyocins are equivalent to the tails of the myophages, one of the major morphotype groups of phages (FIG. 4, Appendix), distinguished by contractile tails and six long tail fibers emanating from a base-plate. The F pyocins are equivalent to the tails of siphophages (FIG. 4, Appendix), which are non-contractile, flexible and have four side tail fibers emanating from a cone tip, which itself has a tail spike. R pyocins and myophages are ideal for engineering adsorption specificity because the receptor-binding domains are located near the tip end of the long tail fibers. Each tail fiber consists of a trimer of the tail fiber protein, which are arranged with the N-terminus proximal to the base plate, so the C-terminal domain of the tail fiber contains the receptor-binding domain. In contrast, siphophages, and by extension, F-type pyocins, have specificity determinants both at the tip of the side tail fibers and also on the spike emanating from the cone. Moreover, although the F-type pyocins are bactericidal, they kill at only at a much higher ratio of pyocin to target cell, suggesting that they are not single-hit lethal.

The genetic determinants of pyocins are, in effect, reduced versions of prophages, the integrated form of lysogenic phages. Each pyocin locus has all the genes for the tail and tail fiber and also genes that are functionally analogous to the repressor systems of lysogenic phage. Moreover, each pyocin locus has a complete lysis gene cassette. The pyocin loci are regulated so that in each generation, a small fraction of the population undergoes spontaneous de-repression, resulting in the expression of the pyocin genes and assembly of complete pyocins, which are then released by lysis of the host. Thus a culture of a pyocin-containing strain of Pseudomonas continuously produces and releases pyocins, which kill other strains of Pseudomonas and, sometimes, strains of other genera. Moreover, massive culture-wide expression of pyocins can be obtained by inducing the SOS response by treating the cells with DNA-damaging agents, as is the case for induction of most lysogenic bacteria.

Conveniently, the widely used laboratory strain Pseudomonas PAO1 produces the R-type pyocin R2, which has a strictly defined host range. Bioinformatic analysis reveals that its tail fiber gene is gene prfl 5. The simplest approach is to set up a plasmid complementation system by knocking out the tail fiber gene on the chromosome and then providing an inducible plasmid-borne copy of the gene. Robust tools for chromosomal gene knockouts and modifications, as well as good inducible plasmid vectors that can be carried in both E. coli and Pseudomonas, are in hand or available. Typically hundreds to thousands of pyocin molecules, or about a microgram of multi-protein structure per ml of culture, are produced and released as a result of cell lysis after induction, and the yield of multi-protein structures is easy to assay both by spot-test killing assays on lawns of susceptible and insensitive target strains, protein gel analysis, and other straightforward physical, biochemical and electron-microscopic methods.

A key strategic point is that, as soon as the complementation system is established, it would be critical to inactivate the lysis system of the pyocin. Because there is always a spontaneous level of induction in pyocin-producing Pseudomonas strains, the cells are continuously exposed to the pyocins in the medium. However pyocin-producing strains are naturally resistant to their own pyocins, presumably because the surface receptor has been altered or eliminated through mutation. Obviously, before we try to make the pyocin host range universal, it is critical to inactivate the lysis genes of the pyocin determinant, a relatively easy task especially for us since identifying and characterizing phage lysis genes has been a major focus of our laboratory. It should be noted that inactivation of the holin, one of the two essential lysis genes, will actually improve multi-protein structure yields, since the induced cell continues to assemble the multi-protein structures long after the normal lysis time programmed into the holin sequence^{B66}.

It is likely that the gene immediately downstream, prf16, encodes a chaperone specific for the tail fiber assembly, based on analogies with tail fibers of T-even phages and lambda, so the complementing clones will be constructed with the putative chaperone genes, to optimize multi-protein structure yields. Once this system is established, the next step is to insert the DNA encoding BPI_{NTD} into the C-terminal segment of the tail fiber gene. The crystal structure of BPI_{NTD} strongly suggests that fusing it to another polypeptide chain is unlikely to interfere with its function, since both the N and C termini of the fragment are directed away from the main fold of the protein. The major unknowns are where to place the heterologous BPI_{NTD} domain, what part of the parental prf15 gene to delete, and whether or not the presence of the domain will interfere with functional folding of the recombinant tail fiber or with the putative chaperone-function of the cognate prf16 protein. However, the DNA manipulations are easy, there are clearly defined predicted secondary structure domains within the tail fiber genes^{T22,T23}, and the biological and biochemical assays are powerful and sensitive. The simplest test for gain of new function is to assay pyocin-killing on lawns of Pseudomonas strains that are naturally resistant to pyocin R2. The killing assays are simple spot tests of dilution series on soft-agar lawns, and are sensitive over many orders of magnitude; even in the case where a construct was marginally stable or poorly folded, we might be able to see a gain of activity against an insensitive target strain and then attempt to optimize the position of the BPI_{NTD} domain. Note that we can easily monitor the production, folding and assembly of each recombinant tail fiber, and even the binding of the recombinant multi-protein structures, independently of the killing function, so we should be able to tell whether we are making progress as we make successive educated guesses for the deletion/substitution of the prf15 gene. Moreover, we would make “positive control” constructions using the tail fibers from homologous phages and pyocins, effectively switching the host range for pyocin R2, which would provide some confidence that the prf15 tail fiber can be parsed into functional domains. There is already precedent in the literature for such swaps. (Williams S R, Gebhart D, Martin D W, Scholl D. Retargeting R-type pyocins to generate novel bactericidal protein complexes. Appl Environ Microbiol. 2008 June; 74(12):3868-76. Epub 2008 Apr. 25).

### Example 2

### Creating and Engineering a Contractile Multi-Protein Structure from Temperate E. coli Myophages

Given the modular nature of most phage genes, it is possible that the trial-and-error approach outlined above has a reasonable chance of working, or at least clearly delineating any potential problems with the design. An initially falsifying result would be that we might obtain recombinant multi-protein structures which have the fully assembled BPI_{NTD}-substituted Prf15 molecules, but that these multi-protein structures either do not bind to the surface of target cells, or do so but do not kill the target cells. Even in that case, it would be instructive to investigate at what level the recombinant fiber fails to function, and to accomplish this a full range of electron microscopic tools are available, including cryo-EM tomography, that would reveal whether or not the distinct steps of irreversible multi-protein structure adsorption and contraction are occurring.

Nevertheless, a parallel approach will also be pursued that would allow us to use the powerful genetic selections characteristic of phage biology. The phages of choice would be the classic, well-studied temperate myophages P2 and Mu, for which a wealth of genetic and molecular information is available^{C25,C30}. This scheme depends on a single assumption: that a temperate myophage can be converted into a contractile multi-protein structure simply by inactivating the head morphogenesis pathway. It has been firmly established that myophage morphogenesis consists of three independent, parallel pathways in vivo: head assembly, tail assembly, and tail fiber assembly^{T18}. The functional phage particle involves head-tail joining and assembly of the six mature tail fibers into slots on the tail base-plate. This means that mutants defective in head formation should be functional multi-protein structures. Oddly, this has not been tested with non-reverting (i.e., deletion-based) head defects in the well-studied temperate myophages P2 and Mu, and it will be a priority to correct this historical oversight. It is possible that additional mutations would be required to make the mature tails sufficiently stable, in the absence of heads, to be robust multi-protein structures. If so, generalized mutagenesis and large-scale screening strategy to obtain these mutations would have to be implemented. However, this is unlikely, because head mutants of several different temperate prophages have been used as “tail-donors” for in vitro phage assembly. Moreover, in one case, it has been shown that a pyocin can complement a tail defect in a Pseudomonas bacteriophage.

Assuming we can demonstrate that either P2 or Mu can be easily converted into a multi-protein structure by head gene inactivation, then the overall scheme is straigtforward. We will set up a plasmid complementation system as described above, where the tail fiber protein is supplied to a tail fiber-less prophage in trans from an inducible plasmid; the only difference is that plaque-formation ability can be followed, rather than killing assays. Mu has an unusual invertible gene cassette that switches the C-terminal half of the tail fiber and thus can generate phage particles with two different host ranges^{C30}. This inversion system would be inactivated for our purposes, but it serves to re-emphasize the fact that the C-terminal receptor-binding regions can be treated as independent folding domains. A number of deletion-BPI_{NTD} substitution constructs will be made in the C-terminal half of the plasmid-borne tail fiber gene and screened for function by plaque-formation on E. coli strains normally resistant to P2 or Mu (both of which recognize LPS decorations as their normal receptors^{C25,C30}). However, if we get weakly positive results with this system, we can optimize by random mutagenesis and selecting for plaque-formers on the resistant lawns. In addition to this powerful selection capability, all of the biochemical, immunological, and ultrastructural assays noted above for the Pseudomonas pyocin system would be available to us with this phage-based system, as well as the wealth of Mu and P2 genetics.

Success will leave us with a P2 or Mu chimera that can make plaques on normally resistant E. coli strains and should be able to absorb to and inject DNA into (but not necessarily kill, replicate in or make plaques on) heterologous Gram-negative bacteria, like Pseudomonas PAO-1. These phages, in their lysogenic form, can then easily be converted to multi-protein structures by deleting genes essential for head morphogenesis. These engineered P2 or Mu-derived multi-protein structures should be able to kill the heterologous Gram-negative bacteria, irrespective of whether the phage version of the same construct could replicate or not.

### Example 3

### Engineering Gram-Positive Multi-Protein Structures

We have identified an innate immunity protein domain (Nod2_{CTD}) that could confer universal host range on a Gram-positive multi-protein structure. It would not be beyond reason that we could substitute Nod2_{CTD} for BPI_{NTD} in our Gram-negative Universal Multi-protein structure and have the new chimera be lethal for Gram-positive bacteria. However, this may not because of the differences in the thickness of the Gram-positive peptidoglycan layer through which the tail tube must protrude to reach the cytoplasmic membrane. In this case, construction of a Universal Multi-protein structure for Gram-positive bacteria much be considered more speculative, for several reasons. First, although the gross morphology of phages of Gram-positive and Gram-negative bacteria is basically the same, much less is known about the process of phage adsorption and DNA injection. Second, no natural multi-protein structures have been reported, although it has been shown that the tails of one Staphylococcus temperate phage can kill the host lysogenic strain. Therefore it will be necessary to convert a temperate myophage to a multi-protein structure by inactivating the head genes, analogously to the process outlined above for P2 and Mu. Third, there may be more variability in the thickness of the Gram-positive cell wall, and thus it may be necessary for contractile phages (and multi-protein structures) to have tails that are of a specific length. Thus using a single temperate myophage might not work for all Gram-positive targets. Unfortunately, almost all temperate phages of Gram-positive cells identified to date are siphophages. A few temperate myophages have been found, including one in Streptococcus. For all of these reasons, efforts to generate Gram-positive multi-protein structures will be started only after success in the Gram-negative experiments.

Advantageously, embodiments disclosed herein may provide engineered phages and multi-protein structures targeted to Gram-negative and Gram-positive bacteria. Multi-protein structures, in particular, are considered to be prime candidates for use as anti-bacterial agents because they have no DNA and are thus less subject to regulatory restrictions than bacteriophages. The constructs disclosed herein may be used in various treatment regimens to remove or substantially reduce bacterial colonies. For example, a surface or aqueous environment, such as water, may be treated with the multi-protein structures, any of the phages, and any of the antibacterial agents disclosed herein.

Although specific embodiments have been disclosed herein in some detail, this has been done solely for the purposes of describing various features and aspects of embodiments, and is not intended to be limiting with respect to the scope of these embodiments. It is contemplated that various substitutions, alterations, and/or modifications, including but not limited to those implementation variations which may have been suggested herein, may be made to the disclosed embodiments without departing from the spirit and scope of the embodiments as defined by the appended claims which follow.

1. A multi-protein structure comprising:
a phage with a chimeric tail fiber protein engineered to replace at least a portion of an endogenous receptor binding domain with a protein domain capable of binding an invariant receptor present on the surface of a bacterial cell, wherein the invariant receptor is selected from the group consisting of Lipid A and L-alanyl-D-glutamyl-N-acetylmuramic acid (MurNac-L-Ala-D-Glu).

2. The multi-protein structure of claim 1, wherein the phage is a headless, lysis-defective phage with a contractile tail.

3. The multi-protein structure of claim 1, wherein the phage is a myophage.

4. The multi-protein structure of claim 3, wherein the myophage is the coliphage P2 or the coliphage Mu.

5. The multi-protein structure of claim 2, wherein the headless, lysis-defective phage with a contractile tail is a pyocin-like bacteriocin.

6. The multi-protein structure of claim 2, wherein said headless, lysis-defective phage with a contractile tail is a pyocin.

7. The multi-protein structure of claim 6, wherein the pyocin is R-type.

8. The multi-protein structure of claim 6, wherein the pyocin is F-type.

9. The multi-protein structure of claim 1, wherein the invariant receptor is Lipid A.

10. The multi-protein structure of claim 1, wherein the invariant receptor is L-alanyl-D-glutamyl-N-acetylmuramic acid (MurNac-L-Ala-D-Glu).

11. The multi-protein structure of claim 1, wherein the protein domain is the amino-terminal domain of a bacteriocidal/permeability-increasing protein (BPI).

12. The multi-protein structure of claim 1, wherein the protein domain is a Nod2 carboxy terminal domain.

13. The multi-protein structure of claim 1, wherein the protein domain is a binding domain of a member of the Pattern Recognition Receptors (PRR) family.

14. A chimeric phage tail fiber protein comprising:
a tail fiber protein engineered to replace at least a portion of an endogenous receptor binding domain with a protein domain capable of binding an invariant receptor present on the surface of a bacterial cell, wherein the invariant receptor is selected from the group consisting of Lipid A and L-alanyl-D-glutamyl-N-acetylmuramic acid (MurNac-L-Ala-D-Glu).

15. A vector capable of expressing a nucleic acid sequence that encodes the chimeric phage tail fiber protein claim 1, wherein said vector comprises said nucleic acid sequence and a regulatory element for expression of said nucleic acid sequence.

16. An isolated bacterial cell comprising the vector of claim 15.

17. The isolated bacterial cell of claim 16, wherein said bacterial cell harbors a prophage.

18. A method of producing an anti-microbial agent, said method comprising culturing the bacterial cell of claim 16 under conditions favoring the growth of the cells.

19. A universal anti-microbial agent comprising the multiprotein structure of claim 1.

20. An antimicrobial agent comprising a phage tail fiber protein, wherein at least a part of a receptor binding domain of said phage tail fiber protein is the amino-terminal of a bacteriocidal/permeability-increasing protein.

21. An antimicrobial agent comprising a phage tail fiber protein, wherein at least a part of a receptor binding domain of said phage tail fiber protein is the carboxyl terminal of a Nod2 receptor protein.

22. A phage tail fiber protein, wherein at least a part of a receptor binding domain of said phage tail fiber protein is the amino terminus of a bacteriocidal/permeability-increasing protein.

23. A phage tail fiber, wherein at least a part of a receptor binding domain of said phage tail fiber is the carboxyl terminus of a Nod2 protein.

24. A method of reducing a bacterial population disposed on a surface, said method comprising exposing the surface to the universal anti-microbial agent of claim 19.

25. A method of reducing a bacterial population in an aqueous environment, said method comprising adding the universal anti-microbial agent of claim 19 to the aqueous environment.
